(19) United States
(12) Patent Application Publication
(10) Pub. No.: US 2014/0144447 A1
(43) Pub. Date: May 29, 2014

(54) CONDUIT

(71) Applicants: Gilbert Jacobus Kuypers, Auckland (NZ); Richard Anthony McCulloch, Auckland (NZ)

(72) Inventors: Gilbert Jacobus Kuypers, Auckland (NZ); Richard Anthony McCulloch, Auckland (NZ)

(21) Appl. No.: 13/882,162

(22) PCT Filed: Oct. 29, 2010
(86) PCT No.: PCT/IB10/54899

§ 371 Date: Jul. 15, 2013

(51) Int. Cl. A61M 16/20; A61M 16/08

(52) U.S. Cl. CPC A61M 16/208; A61M 16/0875
USPC 128/205.24; 128/204.18; 128/205.25

(57) ABSTRACT

A conduit operative or to be operative between a patient interface and both (a) a tidal volume gas delivery device that can deliver controlled tidal volume gasses to a patient for the purposes of resuscitation and (b) a CPAP device for delivery of CPAP to the patient. The conduit comprises a primary lumen having an inlet operatively connectable with said tidal volume gas delivery device. Also provides is a secondary lumen having an inlet operatively connectable with the CPAP device. A manifold, via respective outlets of the primary and secondary lumens and a manifold outlet, allows fluid communication to be established between a patient interface and both the CPAP device and the tidal volume gas delivery device.

### FIELD OF THE INVENTION

The present invention relates to a conduit, more particularly, though not solely, to a respiratory conduit for providing inspiratory gas to a patient interface.

### BACKGROUND TO THE INVENTION

Improvements in conduit for directing respiratory gas or gases to a patient or an interface for the patient (that is a patient interface) are continually sought after. Such conduit is widely used as part of those facilities available as a medical resource in providing respiratory gases to a patient.

More specialised conduit design is employed to allow for greater control of the respiratory gases being supplied to a patient or patient interface especially where accurate tidal volume control is desired for resuscitation applications. Such accuracy being even more important for neonatal applications.

In addition, providing continuous positive airway pressure (CPAP) can be useful depending on a patient's medical state or condition.

Providing an improved conduit capable of supplying both CPAP respiratory gas and accurate delivery of a respiratory gas tidal volume to a patient is advantageous as it can allow the provisioning of both in a single device. But supplying both CPAP respiratory gas and accurate delivery of a respiratory gas tidal volume to a patient has problems because the gasses are compressible and with such compressibility comes a reducing in accuracy, both in pressure and timing, particularly for tidal volume control.

It is therefore an object of the present invention to provide a conduit, and/or a respiratory conduit for providing inspiratory gas to a patient interface which will go at least some way towards addressing the foregoing problems or which will at least provide the industry or public or both with a useful choice.

In this specification where reference has been made to patent specifications, other external documents, or other sources of information, this is generally for the purpose of providing a context for discussing the features of the invention. Unless specifically stated otherwise, reference to such external documents is not to be construed as an admission that such documents, or such sources of information, in any jurisdiction, are prior art, or form part of the common general knowledge in the art.

Further aspects and advantages of the present invention will become apparent from the ensuing description which is given by way of example only.

### BRIEF DESCRIPTION OF THE INVENTION

In a first aspect, the present invention may be said to be a conduit for delivery of inspiratory gas to a patient interface, the conduit comprising:
a primary lumen having an inlet operatively connectable with a primary inspiratory gas supplier, and
a secondary lumen having an inlet operatively connectable with a secondary inspiratory gas supplier,
wherein respective outlets of the primary and secondary lumens terminate in a communal region, the outlet of the secondary lumen including a first pressure responsive valve,
and wherein the communal region is communicable with a patient interface via a second pressure response valve, such that in the second valve's closed position an exhaust port or ports to the surrounding environment become communicable with the patient interface.

Preferably the first valve responsively:
opens (or is open) when the pressure supplied within the secondary lumen is greater than the pressure within the communal region, and
closes (or is closed) when the pressure supplied within the secondary lumen is equal to or less than the pressure within the communal region.

Preferably the second valve responsively:
opens (or is open) when the pressure within the communal region is greater than the pressure within the patient interface, and
closes (or is closed) when the pressure within the communal region is equal to or less than the pressure at the patient interface.

Preferably the second valve, in the open position, allows for delivery of the gases delivered to the communal region to the patient interface.

Preferably the outlet of the primary lumen terminates directly into the communal region.

Preferably the outlet of the primary lumen is open-ended.

Preferably the secondary inspiratory gas supplier is capable of providing a substantially constant pressure gas.

Preferably a blower, optionally an electrically driven blower, provides the substantially constant pressure gas.

Preferably the secondary inspiratory gas supplier provides for a continuous positive airway pressure (“CPAP”) for a patient.

Preferably the primary gas supplier can provide delivery of a controlled tidal volume of gas to a patient interface.

Preferably the primary inspiratory gas supplier is an electrically operable device comprising:
a pump including a rigid cylinder including at least one gas inlet and at least one gas outlet, a piston to travel in the cylinder, and at least one valve, the or each valve configured to allow gas to be displaced into the cylinder through the at least one gas inlet during at least one of a first stroke direction and/or a second stroke direction of the piston in the cylinder, and for allowing gas to be displaced through the at least one gas outlet for delivery to the communal region during an opposite of the at least one of the first stroke direction and/or second stroke direction of the piston in the cylinder,
a motor, selected from one of a stepper motor and feedback motor or a stepper motor with feedback and linear motor, operatively connected to the piston to move the piston in the cylinder.

Preferably the primary inspiratory gas supplier is the electrically operable device as described by PCT/NZ2008/000128 (WO/2008/147229)

Preferably the electrically operable device further comprises of a blower for providing a CPAP source of inspiratory gas to the communal region via the at least one outlet.

Preferably the primary lumen is of smaller cross-sectional area than the cross sectional area of the secondary lumen.

Preferably the primary lumen is of smaller volume than the volume of the secondary lumen.

Preferably the primary and secondary lumens are of the same or substantially similar length from inlet to outlet and/or are preferably coextensive and/or are preferably joined together.

Preferably the primary and secondary lumen are co-axial.

Preferably the primary and secondary lumens are side-by-side or adjacent one another.

Preferably the first and second pressure responsive valves are one-way valves.

Preferably the first and second pressure response valves allow inspiratory gas to be delivered to the communal region and can prevent a patient's exhalation gas from entering the lumens.

Preferably the second valve is actuatable between open and closed positions via one of an electrical or pneumatic controller or control system.

Preferably the patient interface is a face mask.

Preferably the communal region is defined by a manifold housing.

Preferably the primary and secondary lumens are removably engaged to the manifold housing.

In second aspect the present invention may be said to be a conduit for delivery of inspiratory gas to a patient interface, the conduit comprising:
a primary lumen having an inlet operatively connectable with a primary inspiratory gas supplier, and
a secondary lumen having an inlet operatively connectable with a secondary inspiratory gas supplier,
wherein respective outlets of the primary and secondary lumens terminate in a communal region, the outlet of the secondary lumen including a first pressure responsive valve, the first valve responsively:
   opening (or is open) when the pressure within the secondary lumen is greater than the pressure within the communal region, and
   closing (or is closed) when the pressure within the secondary lumen is equal to or less than the pressure within the communal region,
and wherein the communal region is communicable with a patient interface via a second pressure response valve, the second valve responsively:
   opening (or is opens) when the pressure within the communal region is greater than the pressure at the patient interface, and
   closing (or is closed) when the pressure within the communal region is equal to or less than the pressure at the patient interface,
such that when the second valve is in the closed position, an exhaust port or ports to the surrounding environment can become communicable with the patient interface.

Preferably the second valve is in or is caused to move to the closed position when a patient exhales and the exhaust port or ports are in or are open or are caused to open, to causes the flow of gas caused by exhalation to pass out the exhaust port or ports to the surrounding environment.

Preferably when the second valve is in the open position the exhaust port or ports are closed preventing gas flow to the surrounding environment.

In a third aspect the present invention may be said to be a conduit for delivery of inspiratory gas to a communal region comprising:
a primary lumen having an inlet operatively connectable with a primary inspiratory gas supplier, and
a secondary lumen having an inlet operatively connectable with a secondary inspiratory gas supplier,
wherein respective outlets of the primary and secondary lumens terminate in a communal region, the outlet of the secondary lumen including a first pressure responsive valve, and wherein the communal region is communicable with a patient interface via a second pressure response valve, such that in the second valve's closed position an exhaust port or ports to the surrounding environment become or can become communicable with the patient interface.

In a fourth aspect the present invention may be said to be a conduit for delivery of inspiratory gas comprising:
a primary lumen having an inlet operatively connectable with a primary inspiratory gas supplier, and
a secondary lumen having an inlet operatively connectable with a secondary inspiratory gas supplier,
wherein the secondary inspiratory gas supplier is a continuous positive airway pressure (“CPAP”) supply and the primary inspiratory gas supplier provides controlled tidal volume gas delivery for a patient.

In a fifth aspect the present invention may be said to be a patient interface for receiving inspiratory gas from a conduit as claimed above.

In sixth aspect the present invention may be said to be a method for delivering inspiratory gas to a patient interface comprising:

supplying a primary source of inspiratory gas to an inlet of a primary lumen,

supplying a secondary source of inspiratory gas to an inlet of a secondary lumen from a secondary supplier,

the inspiratory gases being delivered to a communal region at the outlets of the respective lumens, the outlet of the secondary lumen controlling delivery of the primary source of inspiratory gas to the communal region, and the communal region controlling delivery of the gases delivered to the communal region to a patient interface.

Preferably a primary gas supplier supplies the primary source of inspiratory gas.

Preferably a secondary gas supplier supplies the secondary source of inspiratory gas.

Preferably a first pressure responsive valve controls delivery of the secondary source of inspiratory gas to the communal region.

Preferably the first valve responsively:
opens (or is open) when the pressure within the secondary lumen is greater than the pressure within the communal region, and
closes (or is closed) when the pressure supplied within the secondary lumen is equal to or less than the pressure within the communal region.

Preferably a second pressure responsive valve controls delivery of the gases delivered to the communal region to the patient interface.

Preferably the second valve responsively:
opens (or is open) when the pressure within the communal region is greater than the pressure within the patient interface, and
closes (or is closed) when the pressure within the communal region is equal to or less than the pressure within the patient interface.

Preferably opening of the second valve allows for delivery of (or delivers) the gases delivered to (or residing within) the communal region to the patient interface.

Preferably the secondary gas supplier includes a third valve that prevents the flow of gases from the secondary lumen into the primary lumen.

In a seventh aspect the present invention may be said to be a method for delivering gases to a patient interface comprising:
ducting a flow of tidal volume of gas or gasses via a primary lumen, to a communal region of a manifold housing, and
ducting a flow of a continuous positive airway pressure (CPAP) of gas or gasses via a secondary lumen to the communal region of said manifold housing,
ducting of the gases in the communal region via a valve to the patient interface when the valve is open, the valve being open when the pressure in the communal region is greater than the pressure at the patient interface, and
preventing the flow of gas or gasses from the communal region to the patient interface by a closure or closed condition of the valve then the pressure in the communal region is less than the pressure at the patient interface.

Preferably when the flow of gas or gasses from the communal region is prevented by virtue of the closure of the valve, exhaust ports (prefereably valved ports) can be presented to allow any equalisation of pressure between the ambient atmosphere and the pressure at the patient interface to occur.

In a further aspect the present invention may be said to be a conduit operative or to be operative between a patient interface and both (a) a tidal volume gas delivery device that can deliver controlled tidal volume gasses to a patient for the purposes of resuscitation and (b) a CPAP device for delivery of CPAP to the patient, the conduit comprising:
a primary lumen having an inlet operatively connectable with said tidal volume gas delivery device, and
a secondary lumen having an inlet operatively connectable with the CPAP device,
a manifold that, via respective outlets of the primary and secondary lumens and a manifold outlet, allows fluid communication to be established between the CPAP device and the tidal volume gas delivery device.

Preferably valves are provided that prevent the flow of tidal volume gasses from entering the secondary lumen and the flow of exhaled gasses from passing into the primary and secondary lumen.

Preferably the at least one valve is provided to allow exhaled gasses to exhaust to the atmosphere.

Preferably a valve is provided to prevent CPAP gas entering the primary lumen.

The term “comprising” as used in this specification means “consisting at least in part of”. When interpreting each statement in this specification that includes the term “comprising”, features other than that or those prefaced by the term may also be present. Related terms such as “comprise” and “comprises” are to be interpreted in the same manner.

This invention may also be said to consist in the parts, elements and features referred to or indicated in the specification of the application, individually or collectively, and any or all combinations of any two or more said parts, elements or features, and where specific integers are mentioned herein which have known equivalents in the art to which this invention relates, such known equivalents are deemed to be incorporated herein as if individually set forth.

The invention consists in the foregoing and also envisages constructions of which the following gives examples only.

### BRIEF DESCRIPTION OF THE DRAWINGS

Preferred embodiments of the invention will be described by way of example only and with reference to the drawings, in which:

FIG. 1 illustrates one embodiment of a conduit according to the invention in connection with a tidal volume supplier and CPAP supplier and a patient interface,

FIG. 2A illustrates the communal region and pressure responsive valves in an inhalation CPAP delivery configuration mode according to the embodiment of FIG. 1,

FIG. 2B illustrates the communal region and pressure responsive valves in an inhalation tidal volume delivery configuration mode according to the embodiment of FIG. 1,

FIG. 2C illustrates the communal region and pressure responsive valves in a configuration mode allowing exhalation of respiratory gases from a patient according to the embodiment of FIG. 1,

FIG. 3 illustrates a second embodiment of a conduit according to the invention in connection with a tidal volume supplier and CPAP supplier and a patient interface, including an actuator for actuating the first pressure responsive valve,

FIG. 4A illustrates the communal region and pressure responsive valves in an inhalation CPAP delivery configuration mode according to the embodiment of FIG. 3,

FIG. 4B illustrates the communal region and pressure responsive valves in an inhalation tidal volume delivery configuration mode according to the embodiment of FIG. 3,

FIG. 4C illustrates the communal region and pressure responsive valves in a configuration mode allowing exhalation of respiratory gases from a patient according to the embodiment of FIG. 3, and

FIG. 5 illustrates the third valve in an alternative position to that shown in FIG. 1.

### DETAILED DESCRIPTION OF THE INVENTION

This invention relates to accurate delivery of tidal volume to a patient interface for fluid connection with a patient that can be overlaid with and/or substituted by the delivery of a continuous positive airway pressure (CPAP) supply to the patient interface. Further, this invention provides for a valving arrangement capable of controlling the delivery of supplied tidal volume respiratory gas and CPAP respiratory gas to a patient via an interface.

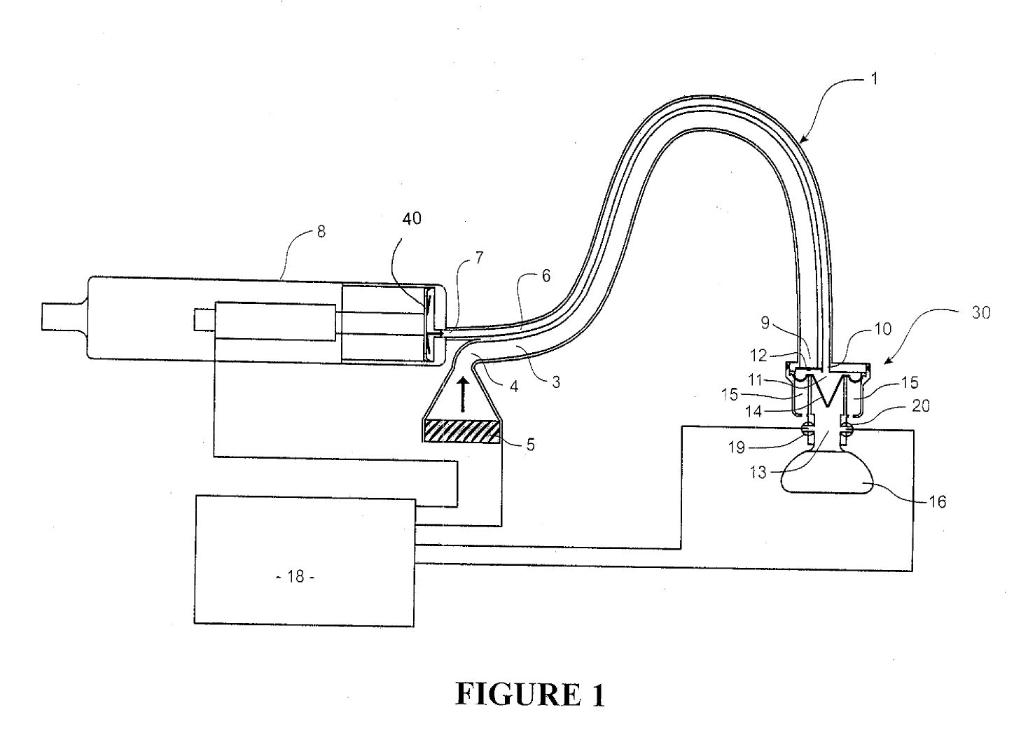

The following description is provided to exemplify the invention with reference to accompanying FIGS. 1-4C.

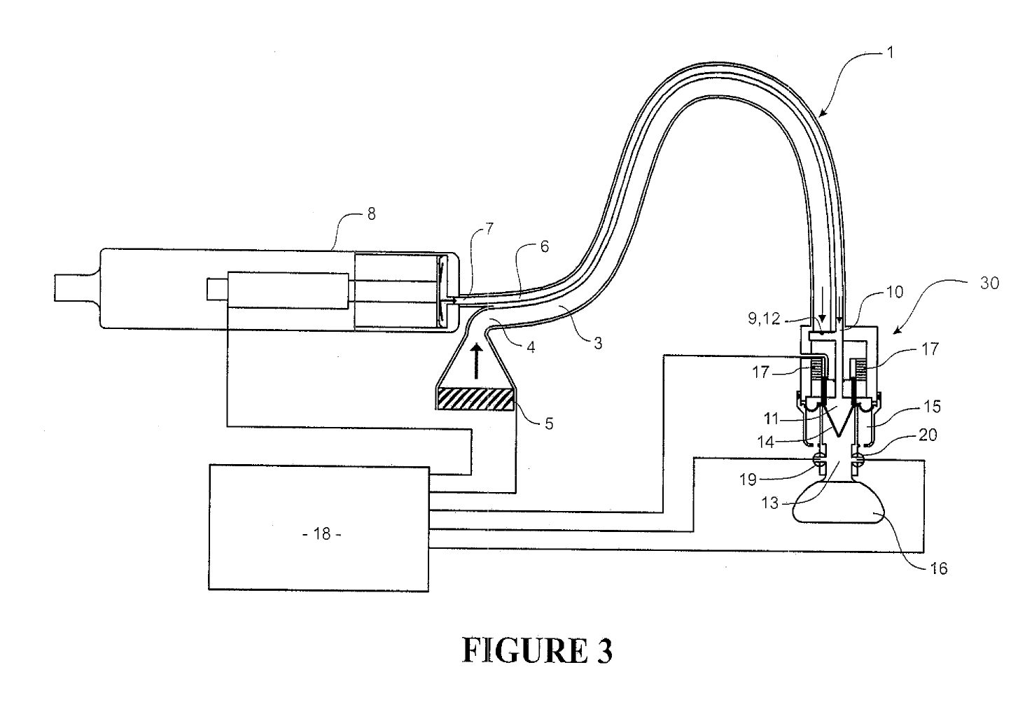

FIGS. 1 and 3 shows a patient interface such as a face mask (16) in ducted fluid communication with a primary inspiratory gas supplier (8) and a secondary inspiratory gas supplier (5). Such may be achieved via lumens (3, 6). Inlets (4, 7) of the lumens (3, 6) are suitably shaped or configured for fluid connection or attachment with the gas suppliers (5, 8) Likewise, at the other end (the patient end), the lumens (3, 6) are suitably shaped or configured for fluid connection with a further device for channelling inspiratory gas to a patient, such as a face mask (16).

According to a first embodiment of the invention, there is provided a conduit (1) for delivery of inspiratory gas to a patient interface such as a face mask (16), the conduit (1) comprising a primary lumen (6) having an inlet (7) operatively connectable with a primary inspiratory gas supplier (8), and a secondary lumen (3) having an inlet (4) operatively connectable with a secondary inspiratory gas supplier (5). Respective outlets (10, 9) of the primary and secondary lumens (6, 3) terminate in a communal region (11). The outlet (9) of the secondary lumen (3) includes a first pressure responsive valve (12). The communal region (11) is able to be, via a second pressure response valve (14), in fluid communication via passage (13) with a patient interface (16). When the second valve is in a closed position an exhaust port or ports (15) may open or be open and allows for the patient interface (16) to be in fluid communication with the surrounding environment.

The communal region (11) and passage (13) in a preferred embodiment are configured within a manifold housing (30). So may the first and second valves (12, 14), the exhaust port(s) 15, be. In this preferred embodiment the primary and secondary lumen (6, 3) are detachable from the manifold housing (30). This allows different sizes or configurations of lumen (6, 3) to be used with the manifold housing (30). It also allows an alternative manifold housing (30) with configurations and/or settings of the manifold's components (such as the first and second valves (12, 14)) to be used with the primary and secondary lumen (6, 3). In this way the conduit can be adapted to suit a particular situation or patient if necessary.

Alternatively the primary and secondary lumen (6, 3) are an integral part and permanently connected to the manifold housing (30).

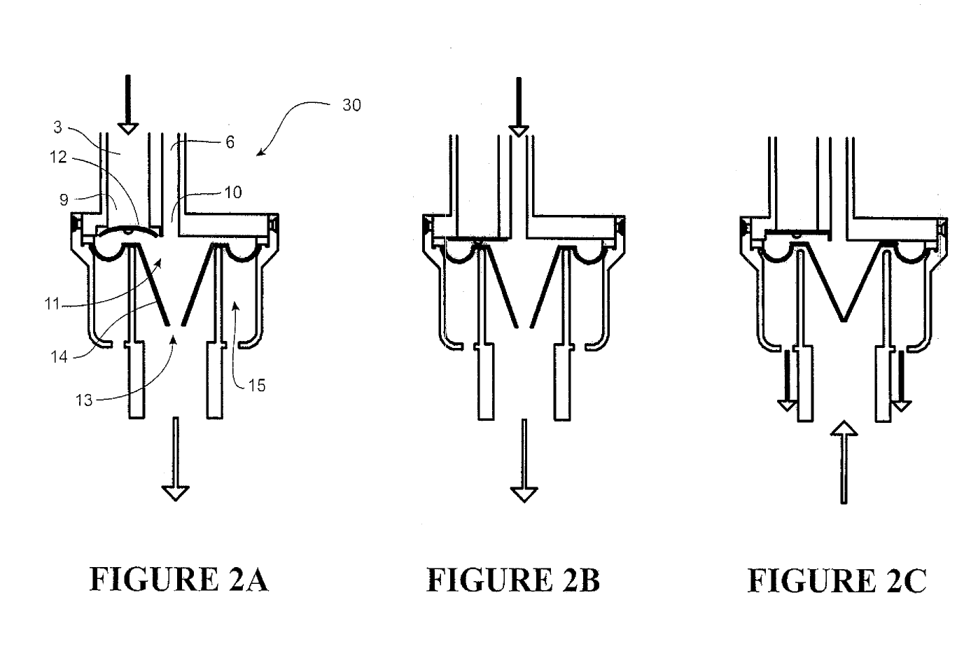

FIGS. 1, 2A, 2B and 2C illustrate a first embodiment of the invention.

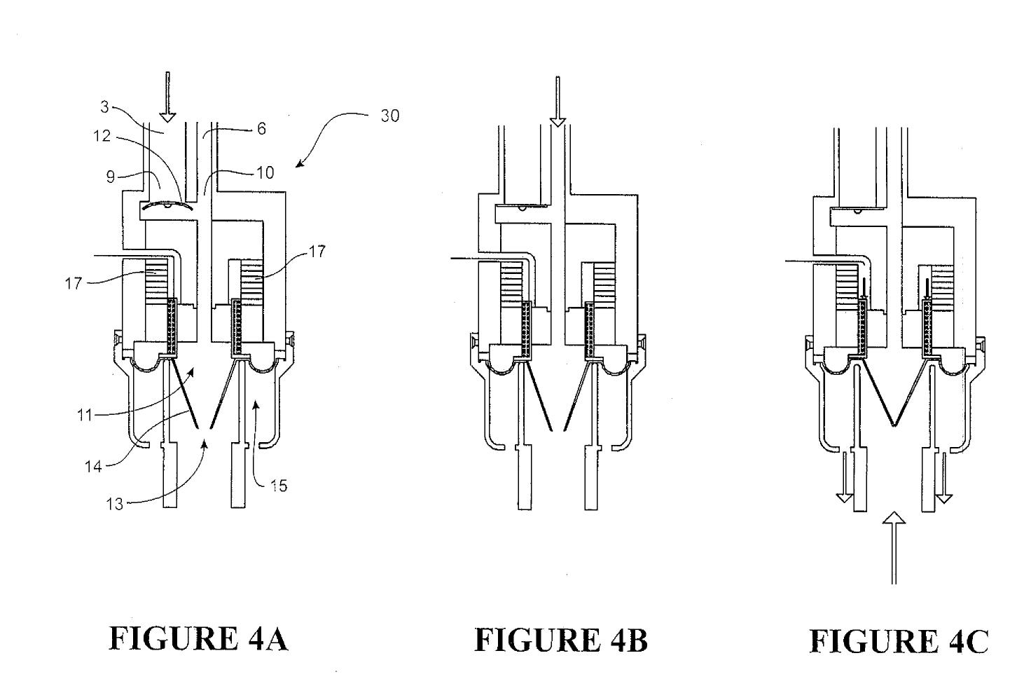

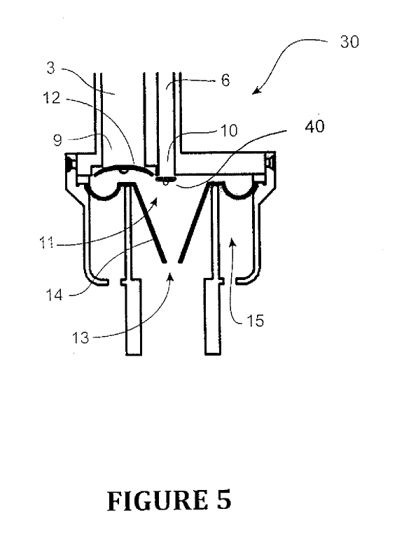

FIGS. 3, 4A, 4B, and 4C illustrate a second embodiment of the invention in which an actuator (17) is included for actuating the second valve (14) between open and closed positions.

Arrows in the figures illustrate direction of flow of inspiratory gas through the lumens (6, 3) from the primary inspiratory gas supplier (8) and secondary inspiratory gas supplier (8), through first valve (12), through the communal region (11) and second valve (14), including any gases caused to pass out of exhaust port or ports (15) when a patient exhales and when the second valve (14) is closed.

In one embodiment the patient interface (16) is a face mask as depicted in FIGS. 1 and 4. In alternative embodiments the patient interface (16) could be any one or more of an endotracheal tube, nasal or nasal-oral cannula or any other apparatus suitable for communicating gases to a patient.

The primary and secondary lumen (6, 3) in one embodiment are detachable from their respective suppliers of gasses.

FIGS. 2B and 4B shows operation of the invention in the resuscitation mode and when CPAP is not being supplied. In this mode, tidal volume is being supplied via the primary lumen (6). FIGS. 2C and 4C show valving configuration during the exhalation stage by a patient, the patient's exhalation gases causing gas to be exhausted via the exhaust port or ports (15) that open in response to closure of the second valve (14). FIGS. 2A and 4A show valving configuration during supply of CPAP gases via the secondary lumen (3) to the patient interface (16). Notably, first valve (12) and second valve (14) are in the open positions allowing CPAP gases and any tidal flow gasses to flow to the patient interface (16).

In one embodiment the primary inspiratory gas supplier (8) includes a third valve (40) that during the supply of CPAP gases only, prevents the flow of gases from the communal region (11) through the primary lumen (6) to the primary inspiratory gas supplier (8). In an alternative embodiment the third valve (40) is located at the outlet (10) of the primary lumen (6).

In various embodiments, the passage (13) allows a ducted fluid communication to be established with the patient interface (16) for a patient (not shown). The patient interface (16) may be removably attached to the passage (13).

According to both the first and second embodiments, the first valve (12) responsively opens or is open when the pressure (P1) supplied within the secondary lumen (3) is greater than the pressure (P11) within the communal region (11), and is closed (or closes) when the pressure (P1) supplied within the secondary lumen (3) is equal to or less than the pressure (P11) within the communal region (11). In this manner, when the first valve (12) is open inspiratory (CPAP) gas can be delivered from the secondary lumen (3) to the communal region (11). Depending on the pressure (P11) within the communal region (11), gas delivered to the communal region (11) may be further delivered to the passage (13).

FIGS. 2A and 4A illustrate the first pressure responsive valve (12) in an open or non-closed position. FIGS. 1, 2B, 2C, 3, 4B and 4C illustrate the first valve (12) in a closed or non-open position.

Further, in both the first and second embodiments, the second valve (14) is responsively open (or opens) when the pressure (P11) within the communal region (11) is greater than the pressure (P13) within the passage (13), and is closed (or closes) when the pressure (P11) within the communal region (11) is equal to or less than the pressure (P13) within the passage (13). In this manner, when the second valve (14) is open inspiratory gas can be delivered from the primary lumen (6) or secondary lumen (3) or both the primary and secondary lumens (6, 3) to the communal region (11) and from the communal region (11) to the patient interface (16). FIGS. 2A, 2B, 4A, 4B illustrate the second valve (14) in an open or non-closed position. FIGS. 1, 2C, 3 and 4C illustrate the second valve (14) in a closed or non-open position.

When the second valve (14) is in the open position the valve (14) allows for further delivery of the gases, being or already delivered to the communal region (11), to further flow to the patient interface (16). In this manner respiratory gases supplied via the primary lumen (6) or secondary lumen (3) or both lumens (6, 3) can be delivered to a patient in a controlled manner.

The outlet (10) of the primary lumen (6) may terminate directly into the communal region (11), as shown by the figures. One particular configuration provides for the outlet (10) of the primary lumen (6) to be open-ended in facilitating this arrangement.

The primary lumen's inspiratory gas supplier (8) provides delivery of a controlled tidal volume source to a patient interface (16) or to a patient, for example via the communal region (11).

The primary inspiratory gas supplier (8) may be an electrically operable device. The electrically operable device may include a pump. The pump may include a rigid cylinder including at least one gas inlet and at least one gas outlet and a piston to travel in the cylinder. At least one valve may be provided, the or each valve configured to allow gas to be displaced into the cylinder through the at least one gas inlet during at least one of a first stroke direction and/or a second stroke direction of the piston in the cylinder, and for allowing gas to be displaced through the at least one gas outlet during an opposite of the at least one of the first stroke direction and/or second stroke direction of the piston in the cylinder. A motor may power the piston. The motor may be a stepper motor or feedback motor or a stepper motor with feedback or linear motor such as a linear servo motor. Such can be operatively connected to the piston to move the piston in the cylinder.

For example, the primary inspiratory gas supplier (8) can be an electrically operable device such as that described by PCT/NZ2008/000128 (WO/2008/147229) the contents of which are hereby incorporated by reference.

The secondary inspiratory gas supplier (5) is a substantially constant pressure source. The secondary lumen's inspiratory gas supplier (5) provides delivery of a continuous positive airway pressure (“CPAP”) source for a patient. For example, the supplier (5) may be a blower, such as an electrically driven blower, providing the substantially constant pressure source. One such suitable blower may be a fan, for example a single phase, 4-poles, brushless DC motor, model no. PSD1204PQBX-A, supplied by Sunonwealth Electric Machine Industry Co., Ltd.

The primary lumen (6) is of a smaller or lesser cross-sectional area than that of the secondary lumen (3). The primary lumen (6) may be of smaller or reduced volume than the volume of the secondary lumen (3). It will be appreciated the primary and secondary lumens (6, 3) may be of the same or substantially similar length, from inlet (7, 4) to outlet (10, 9).

For example, tube sizes are advantageously dissimilar because of the different requirements for tidal volume delivery and CPAP delivery. Tidal volume delivery requires that the respiratory or airway circuit has a minimum amount of dead space (small or lesser cross-sectional or volume of the primary lumen (6)) between the primary inspiratory gas supplier (8), such a pump (for example the pump described in WO/2008/147229), and the patient. Such a configuration assists to minimize the effect of compliance (compressibility of gas) to accurately deliver a predetermined volume of inspiratory gas, such as air/oxygen, to the patient interface (15) and patient (not shown). In general, a pressure range for tidal volume delivery is about a minimum of 0 cm H²O (to ambient air pressure) for exhalation and maximum 60 cm H²O for inhalation. This is even more important for neonatal applications.

In neonatal applications the patients lungs are invariably smaller and as such, smaller tidal volumes of air are required to be delivered. Furthermore, the frequency at which the tidal volumes of air need to be delivered can be much higher for babies than for adult patients. The effects of compliance on the accuracy of the predetermined volume of inspiratory gas delivered are enhanced when smaller tidal volumes and higher frequencies of delivery are required. It is therefore important that the dead space of the respiratory or airway circuit is kept to a minimum by having a small or lesser cross-sectional or volume of the primary lumen (6).

In contrast, CPAP delivery requires that the airway circuit has a low resistance (achieve by providing a large cross-sectional area or diameter of the secondary lumen (3)) between the secondary inspiratory gas supplier (5), such as a blower, and the patient. Such a configuration assists to minimize the effect that resistance has to deliver a constant predetermined airway pressure to the patient. In general, a pressure range for CPAP deliverery is about 0 cm H²O to 30 cm H²O.

The primary and secondary lumens (6, 3) can be co-axial lumens, side-by-side or adjacent lumens. Primary and secondary lumens (6, 3) are shown in a side-by-side arrangement in the accompany FIGS. 1-4C.

The first and second valves (12, 14) are pressure responsive. For example, the valves are responsive to pressure differences between pressure within the respective lumens (6, 3), communal region (11) and at patient interface (16) or passage (13).

The valves (12, 14) may be directional one-way valves. Desirably, the pressure response valves (12, 14) allow delivery of inspiratory gas or gases via the lumens (6, 3) to the communal region (11). The valves can be operated or are operable or responsive for preventing exhalation gas or gases from a patient from entering the communal region (11) or lumens (6, 3).

The pressure responsive valves can be of a flexible valving or membrane type, for example silicon, neoprene or vinyl, suitably soft or flexible to react to the changes in pressure between the secondary lumen (3, P3), communal region (11, P11) and patient interface (13, P13).

A control system (18) can be employed to monitor pressure and flow of gases being delivered to the patient. Such a control system or controller can include one or more pressure sensor(s) (19) and one or more flow sensor(s) (20). Such a control system (18) or controller would be used to control operation of the primary inspiratory gas supplier (8) and secondary inspiratory gas supplier (5). In this manner, a source of tidal volume gas and a source of CPAP gas can be accurately delivered to the primary and secondary lumens (6, 3) for subsequent delivery to the communal region (11) and patient interface (16) and to a patient.

Suitable pressure sensors can be used to measure the airway pressure of the patient which is as close as possible to the patient end of the airway circuit. For example the pressure sensor may be at the patient interface (16) or at the passage (13). Measurements from this sensor can be used in controlling the primary inspiratory gas supplier (8) or ventilator, the secondary inspiratory gas supplier (5) or CPAP, and to open exhaust port or ports (15) (exhaust port or ports (15) and the second valve (14) being actuated by the controller in the second embodiment).

The flow sensor is used to measure the difference between inhalation (device delivered) and exhalation patient returned.

The flow sensor is preferably positioned as close as possible to the patient end of the airway circuit, i.e. at the patient interface (16) or passage (13). Such flow sensor or sensors can be used to check for leakage and the performance of the primary inspiratory gas supplier (8) and can be used to make any necessary or appropriate compensation.

As illustrated by the figures of the second embodiment of this invention (FIGS. 3-4C), the second valve (14) may alternatively be actuatable or be actively controlled to move between open and closed positions via an actuator (17). Such may be by way of an electrical or pneumatic controller that may be operated via the control system (18).

An actuator (17) can for example be included and/or inserted into the system as shown in FIGS. 3-4C between the outlets (10, 9) of primary and secondary lumens (6, 3) in the communal region (11) and between the second pressure valve (14) and patient interface (16) or passage (13). Operation of the conduit and valving system may be the same as the first embodiment but more active control is taken over actuation of the second valve (14). In this manner, the controller and/or operator can have greater active control of the opening and closing of the second valve (14). As the primary inspiratory gas supplier (8), delivers and controls the inhalation pressure part of a patient's breath cycle the actuator (17) and the second valve (14) work to control the exhalation part of the breath cycle of the patient.

The ability to control exhalation pressure is useful for the clinician to be able to set a predetermined shape for the exhalation part of the breath cycle. For example, a clinician may want to retain some back-pressure in the exhalation part of the breath cycle called PEEP (positive end expiratory pressure) or the clinician may want to slow or speed up the exhalation part of breath cycle. The actuator can be controlled by a controller with input from a pressure sensor at the patient interface (16) or passage (13) end of the conduit or respiratory airway circuit.

As a further example, a clinician may want a PEEP of 10 cm H²O, in such a case the controller can be set to 10 cm H²O. When the pressure sensor reads 10 cm H²O (or less) during the exhalation part of the breath cycle the controller powers the actuator (17) to exert a force to operate the second valve (14), the controller then maintains that operation or power until the end of the patient's exhalation cycle. The required force to hold about 10 cm H²O (or other pressure) can be predetermined by the controller and suitable control systems.

In use inspiratory gas to a patient interface (16) may be supplied by supplying a primary source of inspiratory gas to an inlet (7) of a primary lumen (6), supplying a secondary source of inspiratory gas to an inlet (4) of a secondary lumen (3) from a secondary supplier (5), the inspiratory gases being delivered to a communal region (11) at the outlets (9, 10) of the respective lumens (6, 3). A first valve (12) at the outlet (9) of the secondary lumen (3) controls delivery of the secondary source of inspiratory gas to the communal region (11), and the communal region (11) facilitating delivery of the gases delivered to the communal region (11) to a patient interface (16).

In addition, a second pressure responsive valve (14) is used to control delivery of the gases delivered to the communal region (11) to the patient interface (16). In particular, the second valve (14) responsively opens or is open when the pressure (P11) within the communal region (11) is greater than the pressure (P13) within the patient interface (16), and closes or is closed when the pressure (P11) within the communal region (11) is equal to or less than the pressure (P13) within the passage (13) or patient interface (16). It will be appreciated according to the invention described, the opening of the second valve (14) allows for delivery of (or delivers) the gases delivered to (or residing within) the communal region (11) to flow through to the patient interface (16) for subsequent flow to a patient.

Preferably the first and second valves are one way valves that are responsive to pressure differential to effect their opening and/or closing.

It will be appreciated that were reference has been made to a gas or gases that any single gas element or combination of gas elements could be used. It will further be appreciated that were reference is made to a gas or gases that any fluid including liquid, gaseous or a combination of both liquid and gaseous fluids could be used.

### EXAMPLE 1

According to FIGS. 1-2C, the following is a generalised description of operation of the invention.

### 1. CPAP Delivery Stage: Inhalation Part of Patient Airway Cycle (FIG. 2A)

1.1 CPAP flow—a large diameter tube is used for delivery of a secondary inspiratory gas source (5) by an electrically operable blower.
1.2 Large diameter tube is being used for greater control of CPAP airway pressure delivery. This is used to assist in avoiding pressure response problems.
1.3 CPAP airflow pressure delivery is greater than at the end or beginning of tidal volume delivery and higher than patient airway pressure.
1.4 The first valve (12) (valve for CPAP) is open.
1.5 CPAP airflow pressure is then delivered through the first valve (12).
1.6 The second valve (14) is open to allow gas flow through communal region (11) via the passage (13) to the patient interface (16) for delivery to a patient.
1.7 Gas supply or air supplied will not flow back through the primary lumen due to response of the second valve (14) responding to the change in pressure once the patient starts to exhale and the pressure around the patient interface (16) or passage (13) becomes equal to or greater than the pressure in the communal region (11).

### 2. Tidal Volume Delivery Stage: Inhalation (FIG. 2B)

2.1 Tidal volume—smaller diameter tube than the CPAP tube diameter,
2.2 gas delivered by the secondary inspiratory gas supplier, such as an accurate gas volume delivery device. For example by the piston displacement of a secondary inspiratory gas source using the device as described by PCT/NZ2008/000128 (WO/2008/147229).
2.3 The smaller diameter tube is used for greater control of tidal volume delivery to overcome compliance issues.
2.4 Tidal volume airflow pressure is greater than CPAP pressure and patient airway pressure.
2.5 The first valve (12) valve for CPAP gas supply is closed.
2.6 Tidal volume airflow pressure is delivered through the second valve (14) to the patient interface (16).

### 3. Exhalation Stage (FIG. 2C)

3.1 Patient airway pressure is now greater than the tidal volume (primary inspiratory gas supplier (8)) or CPAP (secondary inspiratory gas supplier (5)) pressure.
3.2 The second valve (14) is closed.
3.3 The exhaust ports (15) are open allowing exhalation to surrounding environment.
3.4 Air will not flow back through tidal volume delivery tube (primary lumen (6)) because the second valve (14) is closed.

### EXAMPLE 2

According to FIGS. 3-4C, the following is a generalised description of operation of the invention.

### 4. CPAP Delivery Stage: Inhalation Part of Patient Airway Cycle (FIG. 4A)

4.1 CPAP pressure is set, for example, to 5 cm H²O pressure. This pressure is set at the controller, e.g. at a control panel, and pressure is sensed and checked by a pressure sensor (19).
4.2 CPAP pressure is then delivered through the larger tube of about 10 mm to 15 mm diameter via the CPAP blower device.
4.3 The patient airway has a lower pressure than the set CPAP pressure (P3).
4.4 The first valve (12) being a soft flexible valve made of for example, silicon, opens or is open and delivers air/oxygen to the patient through the now open second valve (14). The second valve (14) is opened by actuation of an actuator (17).

### 5. Tidal Volume Delivery Stage: Inhalation (FIG. 4B)

5.1 The primary inspiratory gas supplier (8) is set as an example to a pressure of 20 cm H₂O and a tidal volume of 25 ml at the controller via a control panel and the pressure is checked by a pressure sensor (19).
5.2 The delivery of the tidal volume is through the smaller tube of about 3 mm to 6 mm diameter.
5.3 The tidal volume pressure is now greater than the set 5 cm H₂O CPAP pressure and the first valve (12) closes the outlet (9) of the larger tube (3). The ventilator continues to deliver air/oxygen through the communal region (11) and through the open second valve (14) to the patient interface (16) until the end of the inhalation delivery cycle. The second valve (14) is kept in the open position by the actuator (17).

### 6. Exhalation Stage (FIG. 4C)

6.1 The patient airway pressure at the patient interface (16) or passage (13) is now greater than either the outlet pressures of the large or smaller tubes (i.e. the primary or secondary lumens (6, 3)) and the pressure in the communal region (11) and greater than the set pressure of 5 cm H₂O CPAP pressure.
6.2 The first valve (12) is closed.
6.3 The patient airway pressure is sensed and the actuator (17) acts to close the second valve (14). As a result of closing the second valve (14) the exhaust ports (15) are opened for release of patient exhalation gases to the surrounding environment allowing the patient to exhale.

The foregoing description of the invention includes preferred forms thereof. Modifications may be made thereto without departing from the scope of the invention.

1. A conduit for delivery of inspiratory gas to a patient interface, the conduit comprising:
a primary lumen having an inlet operatively connectable with a primary inspiratory gas supplier, and
a secondary lumen having an inlet operatively connectable with a secondary inspiratory gas supplier,
wherein respective outlets of the primary and secondary lumens terminate in a communal region, the outlet of the secondary lumen including a first pressure responsive valve,
and wherein the communal region is communicable with a patient interface via a second pressure response valve, such that in the second valve's closed position an exhaust port or ports to the surrounding environment become communicable with the patient interface.

2. The conduit as claimed in claim 1 wherein the first valve responsively:
opens (or is open) when the pressure supplied within the secondary lumen is greater than the pressure within the communal region, and
closes (or is closed) when the pressure supplied within the secondary lumen is equal to or less than the pressure within the communal region.

3. The conduit as claimed in claim 1 wherein the second valve responsively:
opens (or is open) when the pressure within the communal region is greater than the pressure within the patient interface, and
closes (or is closed) when the pressure within the communal region is equal to or less than the pressure at the patient interface.

4. The conduit as claimed in claim 1 wherein the second valve, in the open position, allows for delivery of the gases delivered to the communal region to the patient interface.

5. The conduit as claimed in claim 1 wherein the outlet of the primary lumen terminates directly into the communal region.

6. The conduit as claimed in claim 1 wherein the outlet of the primary lumen is open-ended.

7. The conduit as claimed in claim 1 wherein the secondary inspiratory gas supplier is capable of providing a substantially constant pressure gas.

8. The conduit as claimed in claim 7 wherein a blower, optionally an electrically driven blower, provides the substantially constant pressure gas.

9. The conduit as claimed in claim 1 wherein the secondary inspiratory gas supplier provides for a continuous positive airway pressure (“CPAP”) for a patient.

10. The conduit as claimed in claim 1 wherein the primary gas supplier can provide delivery of a controlled tidal volume of gas to a patient interface.

11. The conduit as claimed in claim 1 wherein the primary inspiratory gas supplier is an electrically operable device comprising:
a pump including a rigid cylinder including at least one gas inlet and at least one gas outlet, a piston to travel in the cylinder, and at least one valve, the or each valve configured to allow gas to be displaced into the cylinder through the at least one gas inlet during at least one of a first stroke direction and/or a second stroke direction of the piston in the cylinder, and for allowing gas to be displaced through the at least one gas outlet for delivery to the communal region during an opposite of the at least one of the first stroke direction and/or second stroke direction of the piston in the cylinder,
a motor, selected from one of a stepper motor and feedback motor or a stepper motor with feedback and linear motor, operatively connected to the piston to move the piston in the cylinder.

12. The conduit as claimed in claim 1 wherein the primary inspiratory gas supplier is the electrically operable device as described by PCT/NZ2008/000128 (WO/2008/147229).

13. The conduit as claimed in claim 11 wherein the electrically operable device further comprises of a blower for providing a CPAP source of inspiratory gas to the communal region via the at least one outlet.

14. The conduit as claimed in claim 1 wherein the primary lumen is of smaller cross-sectional area than the cross sectional area of the secondary lumen.

15. The conduit as claimed in claim 1 wherein the primary lumen is of smaller volume than the volume of the secondary lumen.

16. The conduit as claimed in claim 1 wherein the primary and secondary lumens are of the same or substantially similar length from inlet to outlet and/or are preferably coextensive and/or are preferably joined together.

17. The conduit as claimed in claim 1 wherein the primary and secondary lumen are co-axial.

18. The conduit as claimed in claim 1 wherein the primary and secondary lumens are side-by-side or adjacent one another.

19. The conduit as claimed in claim 1 wherein the first and second pressure responsive valves are one-way valves.

20. The conduit as claimed in claim 1 wherein the first and second pressure response valves allow inspiratory gas to be delivered to the communal region and can prevent a patient's exhalation gas from entering the lumens.

21. The conduit as claimed in claim 1 wherein the second valve is actuatable between open and closed positions via one of an electrical or pneumatic controller or control system.

22. The conduit as claimed in claim 1 wherein the patient interface is a face mask.

23. The conduit as claimed in claim 1 wherein the communal region is defined by a manifold housing.

24. The conduit as claimed in claim 23 wherein the primary and secondary lumens are removably engaged to the manifold housing.

25. A conduit for delivery of inspiratory gas to a patient interface, the conduit comprising:
a primary lumen having an inlet operatively connectable with a primary inspiratory gas supplier, and
a secondary lumen having an inlet operatively connectable with a secondary inspiratory gas supplier,
wherein respective outlets of the primary and secondary lumens terminate in a communal region,
the outlet of the secondary lumen including a first pressure responsive valve, the first valve responsively:
opening (or is open) when the pressure within the secondary lumen is greater than the pressure within the communal region, and
closing (or is closed) when the pressure within the secondary lumen is equal to or less than the pressure within the communal region,
and wherein the communal region is communicable with a patient interface via a second pressure response valve, the second valve responsively:
opening (or is opens) when the pressure within the communal region is greater than the pressure at the patient interface, and
closing (or is closed) when the pressure within the communal region is equal to or less than the pressure at the patient interface,
such that when the second valve is in the closed position, an exhaust port or ports to the surrounding environment can become communicable with the patient interface.

26. The conduit as claimed in claim 25 wherein the second valve is in or is caused to move to the closed position when a patient exhales and the exhaust port or ports are in or are open or are caused to open, to causes the flow of gas caused by exhalation to pass out the exhaust port or ports to the surrounding environment.

27. The conduit as claimed in claim 25 wherein when the second valve is in the open position the exhaust port or ports are closed preventing gas flow to the surrounding environment.

28. A conduit for delivery of inspiratory gas to a communal region comprising:
a primary lumen having an inlet operatively connectable with a primary inspiratory gas supplier, and
a secondary lumen having an inlet operatively connectable with a secondary inspiratory gas supplier,
wherein respective outlets of the primary and secondary lumens terminate in a communal region, the outlet of the secondary lumen including a first pressure responsive valve, and wherein the communal region is communicable with a patient interface via a second pressure response valve, such that in the second valve's closed position an exhaust port or ports to the surrounding environment become or can become communicable with the patient interface.

29. A conduit for delivery of inspiratory gas comprising:
a primary lumen having an inlet operatively connectable with a primary inspiratory gas supplier, and
a secondary lumen having an inlet operatively connectable with a secondary inspiratory gas supplier,
wherein the secondary inspiratory gas supplier is a continuous positive airway pressure (“CPAP”) supply and the primary inspiratory gas supplier provides controlled tidal volume gas delivery for a patient.

30. A patient interface for receiving inspiratory gas from a conduit of claim 1.

31. A method for delivering inspiratory gas to a patient interface comprising:
i. supplying a primary source of inspiratory gas to an inlet of a primary lumen,
ii. supplying a secondary source of inspiratory gas to an inlet of a secondary lumen from a secondary supplier,
iii. the inspiratory gases being delivered to a communal region at the outlets of the respective lumens, the outlet of the secondary lumen controlling delivery of the primary source of inspiratory gas to the communal region, and the communal region controlling delivery of the gases delivered to the communal region to a patient interface.

32. The method as claimed in claim 31 wherein a primary gas supplier supplies the primary source of inspiratory gas.

33. The method as claimed in claim 31 wherein a secondary gas supplier supplies the secondary source of inspiratory gas.

34. The method as claimed in claim 31 wherein a first pressure responsive valve controls delivery of the secondary source of inspiratory gas to the communal region.

35. The method as claimed in claim 34 wherein the first valve responsively:
opens (or is open) when the pressure within the secondary lumen is greater than the pressure within the communal region, and
closes (or is closed) when the pressure supplied within the secondary lumen is equal to or less than the pressure within the communal region.

36. The method as claimed in claim 31 wherein a second pressure responsive valve controls delivery of the gases delivered to the communal region to the patient interface.

37. The method as claimed in claim 36 wherein the second valve responsively:
opens (or is open) when the pressure within the communal region is greater than the pressure within the patient interface, and
closes (or is closed) when the pressure within the communal region is equal to or less than the pressure within the patient interface.

38. The method as claimed in claim 36 wherein opening of the second valve allows for delivery of (or delivers) the gases delivered to (or residing within) the communal region to the patient interface.

39. A method for delivering gases to a patient interface comprising:
i. ducting a flow of tidal volume of gas or gasses via a primary lumen, to a communal region of manifold housing, and
ii. ducting a flow of a continuous positive airway pressure (C-pap) of gas or gasses via a secondary lumen to the communal region of said manifold housing,
iii. ducting of the gases in the communal region via a valve to the patient interface when the valve is open, the valve being open when the pressure in the communal region is greater than the pressure at the patient interface, and
iv. preventing the flow of gas or gasses from the communal region to the patient interface by a closure or closed condition of the valve then the pressure in the communal region is less than the pressure at the patient interface.

40. The method as claimed in claim 39 wherein when the flow of gas or gasses from the communal region is prevented by virtue of the closure of the valve, exhaust ports (prefereably valved ports) can be presented to allow any equalisation of pressure between the ambient atmosphere and the pressure at the patient interface to occur.

41. A conduit operative or to be operative between a patient interface and both (a) a tidal volume gas delivery device that can deliver controlled tidal volume gasses to a patient for the purposes of resuscitation and (b) a CPAP device for delivery of CPAP to the patient, the conduit comprising:
a primary lumen having an inlet operatively connectable with said tidal volume gas delivery device, and
a secondary lumen having an inlet operatively connectable with the CPAP device,
a manifold that, via respective outlets of the primary and secondary lumens and a manifold outlet, allows fluid communication to be established between a patient interface and both the CPAP device and the tidal volume gas delivery device.

42. The conduit as claimed in claim 41 wherein valves are provided that prevent the flow of tidal volume gasses from entering the secondary lumen and the flow of exhaled gasses from passing into the primary and secondary lumen.

43. The conduit as claimed in claim 41 wherein the at least one valve is provided to allow exhaled gasses to exhaust to the atmosphere.

44. The conduit as claimed in claim 41 wherein a valve is provided to prevent CPAP gas entering the primary lumen.
